(19) United States
(12) Patent Application Publication
(10) Pub. No.: US 2007/0106114 A1
(43) Pub. Date: May 10, 2007

(54) ENDOSCOPE-SHAPE MONITORING SYSTEM

(75) Inventors: Hideo SUGIMOTO, Tokyo (JP); Shotaro KOBAYASHI, Tokyo (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/557,510

(22) Filed: Nov. 8, 2006

(30) Foreign Application Priority Data
Nov. 9, 2005 (JP) P2005-324805
Nov. 9, 2005 (JP) P2005-324935
Nov. 9, 2005 (JP) P2005-325226

(51) Int. Cl. A61B 1/00; A61B 5/05

(52) U.S. Cl. 600/117; 600/118; 600/424

(57) ABSTRACT

An endoscope-shape monitoring system is provided that is used to grasp a shape of a flexible insertion portion. The endoscope-shape monitoring system includes a position detecting system, a bending determinator, and a bendable-portion-shape reproducing processor. The position detecting system detects positions of both ends of a bendable portion of the insertion portion. The bending determinator determines a bending situation of the bendable portion. The bendable-portion-shape reproducing processor reproduces the shape of the bendable portion in accordance with the positions and the bending situation.

### BACKGROUND OF THE INVENTION

1. Field of the Invention

The present invention relates to a system or to an apparatus that is used for monitoring the shape of an insertion portion or a flexible tube of an endoscope that is inserted inside a cavity or a hollow of an inspection object.

2. Description of the Related Art

It is beneficial for an endoscopic operator to grasp the shape of a flexible tube of an endoscope that is inserted inside a body. In particular, the visualization of the endoscope shape inside the body has a significant advantage when operating a lower intestinal endoscope, such as a colonoscope, since insertion of the flexible tube into a tortuous intestine is difficult. As a result, various types of endoscope-shape monitoring systems have been proposed.

A system that uses an alternating magnetic field for detecting the shape of a flexible tube of an endoscope is conventionally known. In this system, a plurality of magnetic sensor coils are disposed along the longitudinal direction of the flexible tube, and the three-dimensional position and the direction for each of the coils are detected by using electromagnetic interactions between the alternating magnetic field and the coils. For example, the shape of the flexible tube is represented by a three-dimensional spline curve, which is obtained from positional data of measurement points where the coils are placed, and the result is displayed on a monitor.

The insertion portion of the endoscope generally includes a bendable portion that is connected with a distal end portion, and a flexible portion that connects the bendable portion with an operating portion. The bendable portion is a portion that is bent in connection with an operation of an angle lever provided on the operating portion. On the other hand, the flexible portion is a portion that is flexibly bended.

As schematically illustrated in FIG. 11, the flexible portion 120A is structured from a spiral band member 123, which forms a flexible tube, and the bendable portion 120B is structured from a plurality of bending frame links 121. Each of the neighboring bending frame links 121 is connected together with a hinge section 122, whereby the bendable portion 120B is structured so as to be bendable. Further, an alternative structure of the bendable portion 120B′ is schematically shown in FIG. 12. In the example of FIG. 12, the bendable portion 120B′ includes two types of bending frame links 121A and 1218. In FIG. 12, the bending frame links 121A, which have a narrower width than those of the bending frame links 121B, are applied to the distal end side of the bendable portion 120B′. Therefore, the distal end side of the bendable portion 120B′ can be bent in a wide arc compared to the flexible portion side.

From the structures indicated in FIGS. 11 and 12, the curvatures of the bendable portions 120B and 120B′ when the bendable portions 120B and 120B′ are factitiously bent by an operation of the angle lever 11A are significantly larger than the curvature of the flexible portion 120A, which is due to a flexible bend. Further, the bending manners of the bendable portions 1208 and 120B′ are also quite dissimilar from that of the flexible portion 120A. For example, as shown in FIG. 13, when the bendable portion 120B (120B′) is bent, the bendable portion 120B (120B′) includes a plurality of curvatures whose values are different from one another. Therefore, it is difficult to precisely represent the shape of the bendable portions 120B or 120B′ by applying the same method as used in the representation of the flexible portion 120A.

For the above problems, a system that increases the number of the coils provided on the bendable portion, and densely disposes the coils therein, is provided, so that the shape of the bendable portion is precisely represented.

### SUMMARY OF THE INVENTION

However, when a large number of the coils are provided inside the bendable portion, the permissible range of the bendable portion's curvature becomes limited, so that durability of the bendable portion deteriorates. Further, the number of components and the size of the bendable portion increases.

Therefore, an object of the present invention is to provide an endoscope-shape monitoring system that is able to reproduce the shape of an insertion portion with a relatively simple structure.

According to the present invention, an endoscope-shape monitoring system is provided that is used to grasp the shape of a flexible insertion portion.

The endoscope-shape monitoring system includes a position detecting system, the bending determinator, and a bendable-portion-shape reproducing processor.

The position detecting system detects positions of both sides of a bendable portion of the insertion portion. The bending determinator determines a bending situation of the bendable portion. The bendable-portion-shape reproducing processor reproduces the shape of the bendable portion in accordance with the positions and the bending situation.

According to another aspect of the present invention, an endoscope shape monitoring system that is used to grasp a shape of a flexible insertion portion is provided that includes a distance detector and a memory.

The distance detector detects the distance between both ends of a bendable portion of the insertion portion. The memory stores bendable-portion shape data for reproducing the shape of the bendable portion in accordance with the distance.

### BRIEF DESCRIPTION OF THE DRAWINGS

The objects and advantages of the present invention may be better understood from the following description, with reference to the accompanying drawings in which:

FIG. 1 is a general view of an endoscope to which an endoscope shape monitoring system as a first embodiment of the present invention is applied;

FIG. 2 schematically illustrates an arrangement of coils and a bending sensor provided inside an insertion portion, in the first embodiment;

FIG. 3 is a block diagram that shows overall electrical structures of the electronic endoscope system of the first embodiment;

FIG. 4 indicates a situation where the bendable portion is slightly bent;

FIG. 5 indicates a situation where the bendable portion is bent, where the end face of the distal end portion is turned around by approximately 180 degrees;

FIG. 6 illustrates an example of an image representation of the shape of the insertion portion where the points P1-P8 are connected by segments (a linear interpolation);

FIG. 7 illustrates an example of an image representation of the shape of the insertion portion, where the points P1-P8 form the basis of a Bézier curve or a spline curve;

FIG. 8 indicates the positions of the points P1-P4 and the representation of the linear interpolation thereof, where the bendable portion 12B is bent in a narrow arc;

FIG. 9 schematically illustrates actual shapes of the bendable portion in several bending situations and relations of the positions between the point P1 and the point P2 in each of the bending situations;

FIG. 10 is a graph that schematically represents the relations between the curvature “ρ” and the resistance “R”, as a example;

FIG. 11 schematically illustrates an example of prior art structures of a bendable portion and a flexible portion;

FIG. 12 schematically illustrates another example of prior art structures of the bendable portion and the flexible portion;

FIG. 13 schematically shows the shape of the prior art bendable portion that is bent by a plurality of curvatures;

FIG. 14 schematically illustrates an arrangement of coils and bending sensors provided inside the insertion portion, in a second embodiment;

FIG. 15 is a partially magnified view of a cross section of the bending frame link, in a plane perpendicular to the axis of the bending frame link;

FIG. 16 is a block diagram that shows overall electrical structures of the electronic endoscope system of the second embodiment;

FIG. 17 schematically illustrates positions P1-P5 of the coils S1-S5 and an interpolation curve, when the bendable portion is bent, in which the end face of the distal end portion is turned around by approximately 270 degrees;

FIG. 18 schematically illustrates structures of a sensor unit used in the endoscope-shape monitoring system of the third embodiment;

FIG. 19 is a block diagram that schematically illustrates the endoscope-shape monitoring system of the third embodiment; and

FIG. 20 schematically illustrates the relations between the positional coordinate data (X1,Y1,Z1)-(X9,Y9,Z9) and the bendable portion in situations where the point P1 is positioned at P1(0), PT(4), and P1(8).

### DESCRIPTION OF THE PREFERRED EMBODIMENTS

The present invention is described below with reference to the embodiments shown in the drawings.

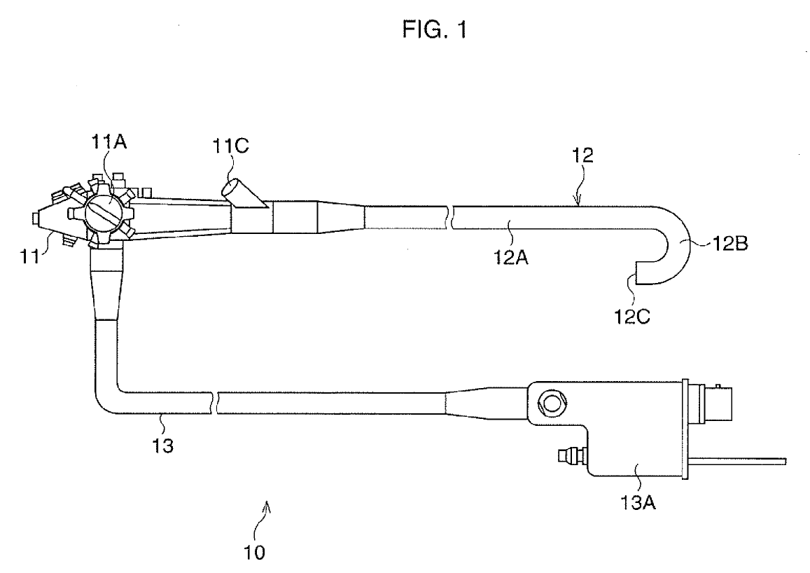

FIG. 1 is a general view of an endoscope to which a first embodiment of an endoscope-shape monitoring system of the present invention is applied. In this embodiment, an electronic endoscope is employed as an example for the endoscope.

The electronic endoscope 10 has an operating portion 11, which an endoscopic operator manipulates. An insertion portion (a flexible tube) 12 and a light-guide cable 13 are both connected to the operating portion 11. A connector 13A is provided at the distal and of the light-guide cable 13. The connector 13A is detachably attached to a processor apparatus (not depicted); for example, in which a light source and an image-signal processing unit are integrally installed. Namely, illumination light from the light source inside the processor apparatus is supplied to a cavity or to a hollow viscus through the connector 13A of the electronic endoscope 10 and the light-guide cable 13. Further, image signals from the electronic endoscope 10 are supplied to the image-signal processing unit inside the processor apparatus.

The insertion portion 12 is comprised of a flexible portion 12A, a bendable portion 12B, and a distal end portion 12C. Most of the insertion portion 12 is occupied by the flexible portion 12A that is formed of a flexible tube, which is freely bendable, and the flexible portion 12A is directly connected to the operating portion 11. The bendable portion 12B is provided between the distal end portion 12C and the flexible portion 12A, and is bended in accordance with a rotational operation of an angle lever 11A that is provided on the operating portion 11. For example, the bendable portion 12B can be bended such that the direction of the distal end portion 12C is rotated by 180 degrees. Further, as will be detailed later, the distal end portion 12C is provided with an imaging optical system, an imaging device, an illuminating optical system, and other components.

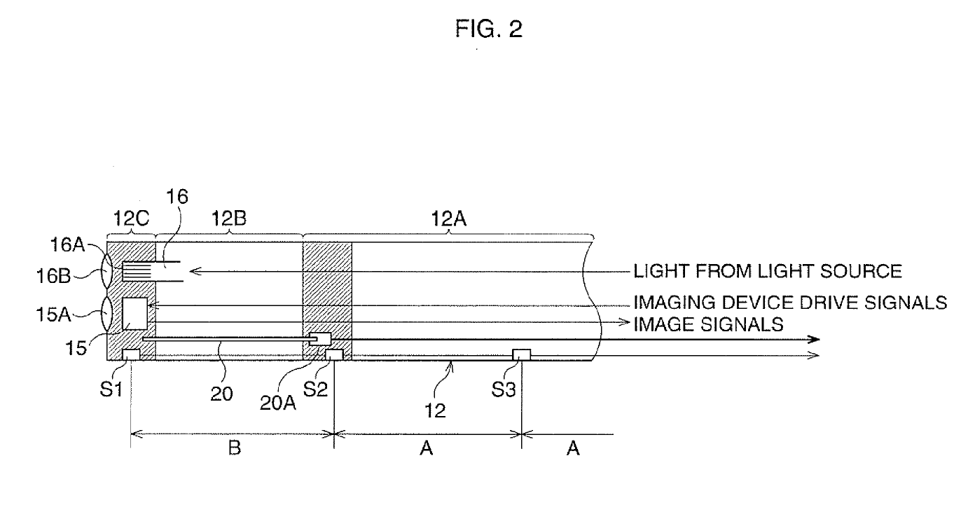

FIG. 2 is a partially magnified view that schematically illustrates the configuration around the bendable portion 12B of the insertion portion 12.

The distal end portion 12C of the insertion portion 12 is formed as a rigid section. Inside the distal end portion 12C, an imaging device 15 and the front end 16A of a light guide (optical fiber bundle) 16 are disposed. Further, an illuminating optical system 16B for emitting light from the light guide 16, and an imaging optical system 1SA for projecting an object image onto the imaging device 15 are also provided in the distal end portion 12C of the insertion portion 12.

Further, a first coil S1 is provided in the distal end portion 12C, and a second coil S2 is provided near the boundary between the bendable portion 12B and the flexible portion 12A. In the present embodiment, the second coil S2 is provided in the flexible portion 12A at a position near the bendable portion 12B. A third coil S3, a fourth coil S4, a fifth coil S5, . . . , and an n-th coil Sn, are successively arranged along the axis of the flexible portion 12A at predetermined intervals “A”, from the side of the coils S2 to the side of the operating portion 11. The first coil Si to the n-th coil Sn are used as magnetic sensors. In FIG. 2, only the coils S1-S3 are indicated as examples. Further, although the bending frame links, as is present in conventional structures, are not depicted in FIG. 2, a suitable bending frame link mechanism is applied to the embodiment.

Further, the bendable portion 12B is provided with a bending sensor 20 that extends along the axis of the bendable portion 12B from the flexible portion 12A to the distal end portion 12C. The bending sensor 20 is a sensor that detects the degree of bending of the bendable portion 12B. In the present embodiment, a strain gauge is adopted. Note that, one end of the strain gauge 20 is fixed to the end of the flexible portion 12A, which is connected to the bendable portion 12B, by a fixing member 20A, while the other end is fixed to the distal end portion 12C.

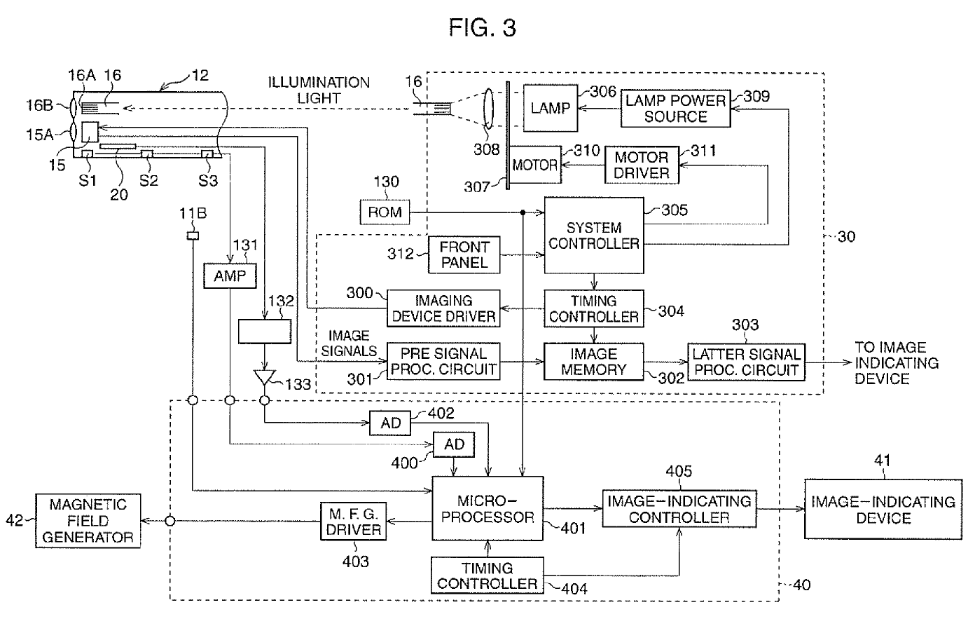

FIG. 3 is a block diagram that shows an electrical structure of the electronic endoscope system of the present embodiment. The electronic endoscope system of the present embodiment includes an insertion-portion-shape monitoring system that detects positions of the insertion portion 12 and indicates the shape thereof, and an capturing-image indicating system that captures an endoscopic image at the distal end of the insertion portion 12 and indicates the captured image.

The capturing-image indicating system generally includes the imaging device 15 and the light guide 16 that are provided inside the insertion portion 12 a processor unit 30, and an image-indicating device (not shown) for indicating an image captured by the imaging device 15. The processor unit 30 supplies illumination light to the light guide 16, drives the imaging device 15, and processes the image signals from the imaging device 15.

On the other hand, the insertion-portion-shape monitoring system generally includes the plurality of coils S1-Sn, which are used as magnetic sensors and provided inside the insertion portion 12 of the endoscope, an insertion-portion-shape monitoring unit 40, an image-indicating device 41 for indicating the shape of the insertion portion 12, and a magnetic field generator 42.

In the present embodiment, the processor unit 30 and the insertion-portion-shape monitoring unit 40 are provided inside the processor apparatus to which the connector 13A (see FIG. 1) is detachably attached. Namely, the signal wires of the imaging device 15, the light guide 16, the signal wires of the coils S1-Sn, and the signal wires of the strain gauge 20 are led to the processor apparatus via the light guide cable 13 (see FIG. 1) and the connector 13A

The light guide 16 and the signal wires of the imaging device 15 are connected to the processor unit 30 provided inside the processor apparatus. The imaging device 15 is driven by an imaging device driver 300 provided inside the processor unit 30, and the image signals from is the imaging device 15 are fed to a pre-signal processing circuit 301 of the processor unit 30.

The image signals that are subjected to predetermined image-signal processes in the pre-signal processing circuit 301 are temporarily stored in an image memory 302, and are then successively fed to a latter signal processing circuit 303. In the latter signal processing circuit 303, the image signals are subjected to predetermined image-signal processes, and then the image signals are encoded as video signals. The video signals are fed to an output device, such as the image-indicating device.

Note that the imaging device driver 300 and the image memory 302 are driven by control signals from a timing controller 304, and a system controller 305 controls the timing controller 304.

Further, the imaging device 15 captures images inside the body, while emitting illumination light from the light guide 16. The illumination light is supplied from the light source unit inside the processor apparatus to the light guide 16. The light source unit includes a lamp 306, and white light from the lamp 306 is concentrated upon the end face of the light guide 16 (which is inserted inside the processor apparatus) via a shutter 307 and a condenser lens 308.

The lamp 306 receives electric power from a lamp power source 309. A motor 310 that is control: ed by a motor driver 311 drives the shutter 307. The lamp power source 309 and the motor driver 311 are controlled by the system controller 305.

Note that the system controller 305 is connected to a front panel 312, which includes switches that are operated by a user. The system controller 305 is able to change various types of preset parameters and modes according to operations of the switches on the front panel 312.

Further, a ROM 130 is provided inside the connector 13A of the electronic endoscope 10. When the connector 13A is attached to the processor apparatus, the ROM 130 is connected to the system controller 305, so that electronic endoscope identification information stored in the ROM 130 is transmitted to the system controller 305. Namely, the ROM 130 stores information relating to the electronic endoscope 10, such as the type of the scope and parameters used in the image processing, and the information is acquired by the system controller 305.

For example, signals from the coils (magnetic sensors) S1-Sn are fed to a multi-channel A/D converter 400 inside the insertion-portion-shape monitoring unit 40 via a multi-channel amplifier 131, and amplified by a predetermined gain. Signals from the coils S1-Sn, which are converted from analog signals to digital signals at the multi-channel A/D converter 400, are input to a microprocessor 401, and the position of each coil S1-Sn is calculated.

On the other hand, variation in electrical resistance in the strain gauge 20 is detected by a strain gauge circuit 132 that is provided inside the connector 13A. Signals that represent the variation in resistance are fed to an A/D converter 402 inside the insertion-portion-shape monitoring unit 40, via a buffer 133 provided inside the connector 13A. Namely, the signals from the strain gauge 20 are converted to digital signals at the A/D converter 402, and are then input to the microprocessor 401.

Further, in the present embodiment, an angle lever sensor 11B for detecting a direction of the angle lever operation (a rotational direction) is provided on the angle lever 11A, which is mounted on the operating portion 11. The angle lever sensor 11B is connected to the microprocessor 401 via signal wires that are wired inside the light guide cable 13 and the connector 13A, so that the signals that are detected by the angle lever sensor 11B are input to the microprocessor 401.

Image data for representing the entire shape of the insertion portion 12 are generated at an image-indicating controller 405, based on the positional data of the coils S1-Sn, which are calculated by the microprocessor 401, the data detected by the strain gauge 20, and the signal from the angle lever sensor 11B. The signals of the image data are then fed to the image-indicating device 41. The image data may represent the shape of the insertion portion 12 by using an interpolation curve line that connects the positions of the coils S1-Sn.

As is known in the prior art, the positions of the coils S1-Sn are obtained by detecting the effects of electromagnetic interactions with the coils S1-Sn, where the effects are induced by the alternating magnetic field. For example, the magnetic field generator 42 generates alternating magnetic fields in turn for each of the X, Y, and Z coordinates of an orthogonal coordinate system XYZ. The magnetic field generator 42 is controlled by a magnetic field generator driver 403. Further, the microprocessor 401, the image-indicating controller 405, and the magnetic field generator driver 403 are all controlled by the timing controller 404.

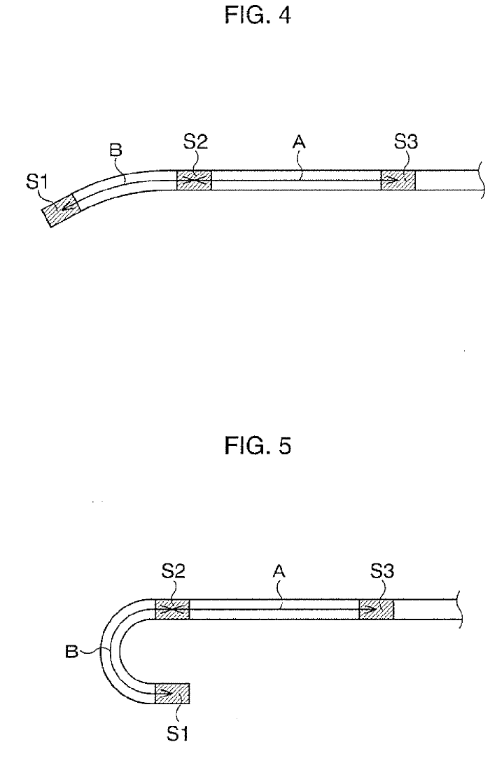

With reference to FIGS. 4-9, the processes for indicating the shape of the insertion portion, in the present embodiment, are described below.

FIGS. 4 and 5 schematically illustrate the shapes of the endoscope insertion portion 12 around the distal end portion, when the angle lever 11A is operated and the bendable portion 12B is bent. FIG. 4 indicates a situation where the bendable portion 123 is slightly bent. FIG. 5 indicates a situation where the bendable portion 12B is bent such that the end face of the distal end portion 12C is turned around approximately 180 degrees.

In the present embodiment, the first coil S1 is provided in the distal end portion 12C of the insertion portion 12. The second coil S2 is disposed in the flexible portion 12A, next to the bendable portion 12B. Further, the second coil S2 is separated from the coil S1 by a distance “B” along the axis. In addition, the coils S3, . . . ,Sn are successively arranged at the predetermined intervals “A”, from the side of the coil S2 to the side of the operating portion 11.

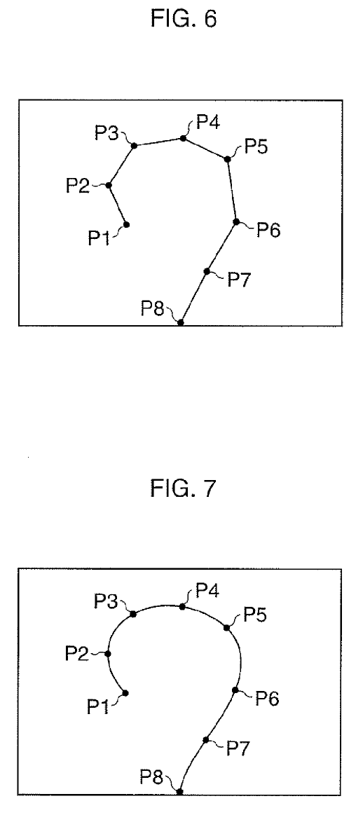

In the insertion-portion shape-indicating process, the shape of the insertion portion 12 is reproduced on the screen of the image-indicating device 41 by connecting the points P1-Pn that correspond to the positions of the coils S1-Sn, where the positions are obtained by using the alternative magnetic field. In FIG. 6, an example of image indication where the points P1-Pn are connected by segments (a linear interpolation) is illustrated. In FIG. 7, an example of image indication where the points P1-Pn are connected or fitted by a Bézier curve or a spline curve is illustrated.

However, the structures of the bendable portion 123 are generally different from those of the flexible portion 12A. Further, the way force acts on the bendable portion 123 is also different from the way force acts on the flexible portion 12A, since the bendable portion 12B is affected by the force of the angle wires. Therefore, the manner of bending of the bendable portion 12B is quite different from that of the flexible portion 12A, so that if the same interpolation method were used for the flexible portion 12A and the bendable portion 12B, as is done conventionally, the reproduced shape of the bendable portion 123 could result in a quite different shape from the actual shape.

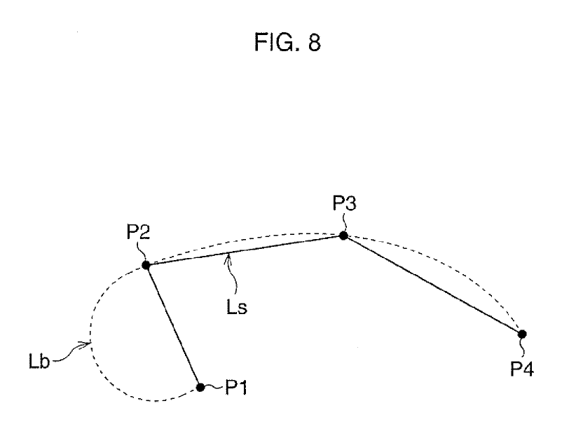

Referring to FIG. 8, the positions of the points P1-P4 and the representation of the linear interpolation thereof, when the bendable portion 12B is bent in a narrow arc, are indicated, Namely, the reproduced shape of the insertion portion 12, which is represented by linear interpolation (where the points P1-P4 are connected by the segments), is described by the solid line Ls. On the other hand, the actual shape of the insertion portion 12 is described by the phantom line Lb.

As shown in FIG. 8, since the flexible portion 12A forms a gentle curve when it is bent, the reproduced shape (Ls) approximates the actual shape (Lb) for the intervals between the points P2-P4 that correspond to the flexible portion 12A. However, for the interval between the point P1 and the point P2 that corresponds to the bendable portion 12B, the reproduced shape is far from the actual shape. As an example of an extreme case, FIG. 8 represents the linear interpolation case. However, even by applying a Bézier curve or a spline curve for the interpolation, it would be difficult suitably to represent the shape of the bendable portion 12B when the bendable portion 12B is bent in a narrow arc, if the same interpolation method were used to represent the flexible portion 12A and the bendable portion 128.

In order to reproduce the shape of the bendable portion 12B accurately, a plurality of magnetic sensor coils may be disposed inside the bendable portion 12B. However, a bending operation due to the manipulation of the angle lever 11A would be obstructed if a coil were disposed inside the bendable portion 12B, and the coil could also be damaged or destroyed. Accordingly, in the present embodiment, the coil S1 and the coil 32 are disposed on both ends of the bendable portion 123, and the strain gauge 20 is disposed in the bendable portion 12B.

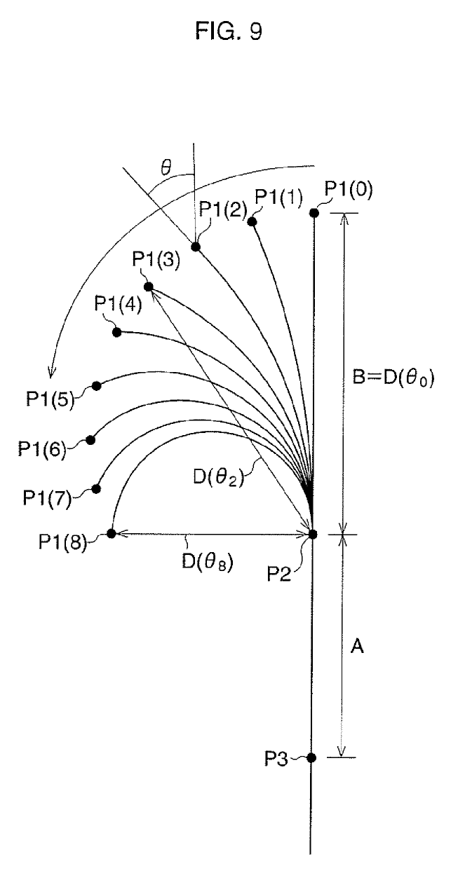

In general, the bending properties of the bendable portion 12B are specific for each product. The actual shapes of the bendable portion 123 in several bending situations, and the relation of the positions between the point P1 and the point P2 in each of the bending situations, are schematically illustrated in FIG. 9. In FIG. 91 nine types of bending situations of the bendable portion 12B are illustrated in stages from the non-bending situation to the situation when the bendable portion 12B is approximately turned around in the opposite direction.

In FIG. 9, the positions of the point P1 in each of the above nine bending situations are represented by P1(0)-P1(8). Further, the direction of the distal end portion 12C when the bendable portion 12B is being bent is represented by an angle “θ”, where the angle troll represents an angle against the direction of the distal end portion 12C, when the bendable portion 12B is directed straight forward and is not bent. Thus, the bending situation is represented by the angle “θ”, Namely, when the bendable portion 12B is not bent and the point P1 is positioned at P1(0), the angle θ=0°. Further, when the bendable portion 12B is bent such that the distal end portion 12C faces in the opposite direction, and when the point P1 is positioned at P1(8), the angle θ=180°. Moreover, the angles “θ” for each of the positions P1(0)-P1(8) are represented by θ0-θ8.

For example, if the curvature of the bendable portion 123, the positions of the points P1 and P2, and the direction in which the bendable portion 12B is bent are all determined, the shape of the bendable portion 12B can be precisely reproduced. Therefore, in the present embodiment, the positions of the coils S1 and S2 (the points P1 and P2) are calculated as described above, and the curvature of the bendable portion 123 is derived from the data obtained by the strain gauge (the bending sensor) 20. Further, the bending direction is detected by the signals from the angle lever sensor 11B provided on the angle lever 11A, so that the precise shape of the bendable portion 123 is reproduced and indicated.

Note that, as is well known in the art, the strain gauge 20 generally is structured such that a resistor element, such as a wire gauge, is attached to a base (a thin plate of electrical insulating material). Namely, deformation of a measurement object is detected by detecting variation in the resistor element's electrical resistance induced by the deformation.

For example, in the present embodiment, the correspondence between the electrical resistance “R” of the strain gauge 20 and the curvature “ρ” of the bendable portion 12B is measured beforehand, and the information thereof is stored in a ROM 130 (see FIG. 3), which is provided inside the connector 13A of the electronic endoscope 10, before shipment. Namely, when the connector 13A of a certain electronic endoscope is attached to the processor apparatus, the above data are transmitted from the ROM 130, with the identification number of the endoscope, to the microprocessor 401.

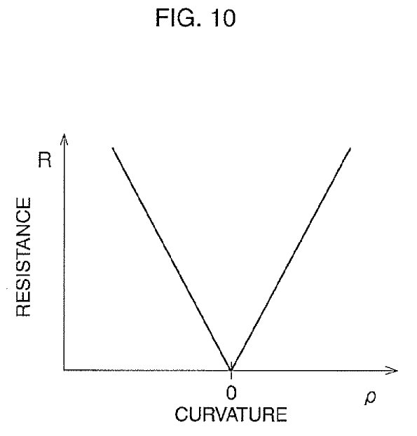

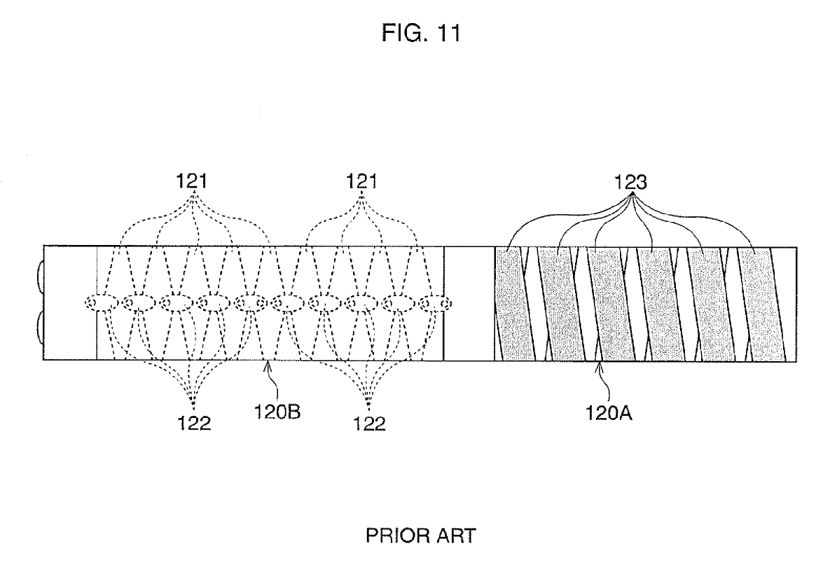

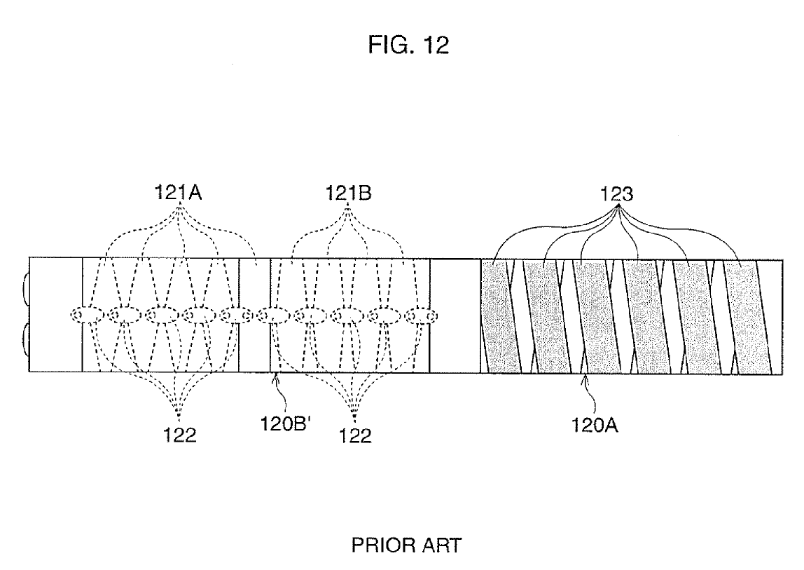

FIG. 10 is an example of a graph that schematically represents the relation between the curvature “ρ” and the electrical resistance “R”. Further, in FIG. 10, whether the curvature “ρ”, is positive or negative is determined by a signal from the angle lever sensor 11B.

As described above, according to the first embodiment, the shape of the insertion portion 12 is reproduced by applying the different methods for the bendable portion 12B and the flexible portion 12A, respectively, so that the entire shape of the insertion portion 12 is more accurately reproduced by the combination thereof. Namely, as for the flexible portion 12A, each position of the coils is connected together with a Bézier curve or a spline curve, in the same way as conventionally way. On the other hand, as for the bendable portion 12B and the distal end portion 12C, the shape is reproduced based on the positions of the first and second coils S1 and S2 (both end positions of the bendable portion), the bending direction of the bendable portion 12B is detected by the angle lever sensor 11B, and the curvature of the bendable portion 12B is obtained from the data of the strain gauge 20.

Note that, when the Bézier curve or the spline curve is used to represent the flexible portion 12A, a control point for the point P2 of the interpolation curve of the flexible portion 12A is determined from the geometrical parameters, such as for the tangential line and the curvature, for the interpolation curve selected for the bendable portion 12B.

As described above, according to the first embodiment, the shape of a bendable portion can be reproduced more precisely with a simple structure, so that the entire shape of the insertion portion can be represented more precisely.

Although the number of the bending sensors (e.g., the strain gauges) is one in the first embodiment, the number of the bending sensors may be a plurality.

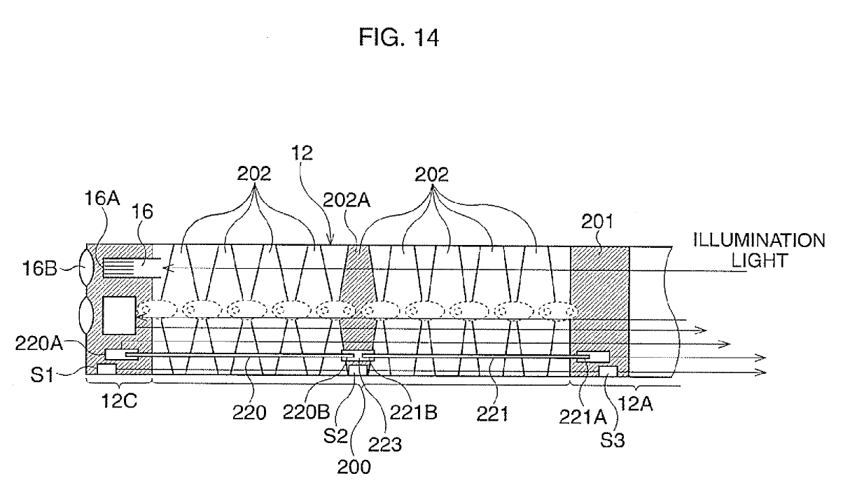

Next, with reference to FIG. 14 to FIG. 17, an endoscope, to which a second embodiment of an endoscope-shape monitoring system of the present invention is applied, is explained below. Although the structures of the second embodiment are dissimilar from those of the first embodiment regarding structures relating to a bending detection, the remaining structures are the same as those in the first embodiment. Therefore, the explanations will mainly be given for the dissimilar structures, and the same reference numerals will be used for the same structures, as those in the first embodiment.

FIG. 14 is a partially magnified view that schematically illustrates the configuration around the bendable portion 200 of the insertion portion 12 of the second embodiment.

As shown in FIG. 14, a ring-shaped rigid section 201 is provided at the boundary between the bendable portion 200 and the flexible portion 12A. A plurality of bending frame links 202 are provided inside the bendable portion 200, as is known in the prior art, so that the bending frame links 202 are successively connected with each other from the distal end portion 12C to the rigid section 201 as a chain.

Further, the coil S1 is provided in the distal end portion 12C, and the coil S2 is provided in a bending frame link 202A (a bending frame link that is hatched in FIG. 14) that is positioned approximately at the midsection of the bendable portion 200. Further, the coil S3 is provided in the rigid section 201. The coils S4, S5, S6, . . . , Sn, are successively arranged along the axis of the flexible portion 12A at predetermined intervals, from the side of the coils 83 to the side of the operating portion 11. In FIG. 14, only the coils S1-S3 are indicated as an example.

In the second embodiment, bending sensors 220 and 221, which are used to detect a bending state of the bendable portion 200, are provided inside the bendable portion 200 along the axis thereof. The bending sensors 220 and 221 is are sensors that detect a bending degree of the bendable portion 200, and in the present embodiment, a strain gauge is used, as in the first embodiment. Note that one end of the strain gauge 220 is fixed to the distal end portion 12C by a fixing member 220A, and one end of the strain gauge 221 is fixed to the rigid section 201.

On the other hand, the other end 220B of the strain gauge 220, which is on the side opposite from the fixing member 220A, and the other end 221B of the strain gauge 221, which is on the side opposite from the fixing member 221A, both extend to the bending frame link 202A. Further, the ends 220B and 2213 engage with the bending frame link 202A through a guide member 223, whereby the ends 220B and 2212 are only slideable along the axis of the bendable portion 200.

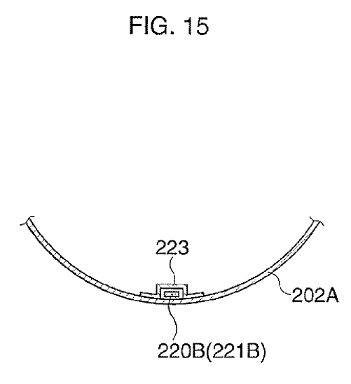

Namely, as shown in FIGS. 14 and 15, the guide member 223 that extends along the axis of the bending frame link 202A is provided on the inner side face of the bending frame link 202A, whereby movement of the ends 220B and 221B other than the movement along the axis of the bendable portion is restricted. On either side of the guide member 202A, in the longitudinal direction, there is provided an opening into which the corresponding end 220B or 221B is inserted. Namely, the ends 220B or 221B are each inserted into the corresponding side of the guide member 202A. Further, in the second embodiment, the ends 220B and 221B are separately disposed at a predetermined distance, whereby they do not come into contact with each other. Note that FIG. 15 is a partially magnified view of a cross section of the bending frame link 202A, in a plane perpendicular to the axis of the bending frame link 202A. Namely, FIG. 15 schematically illustrates the relations between the ends 2203, 221B, and the guide member 223.

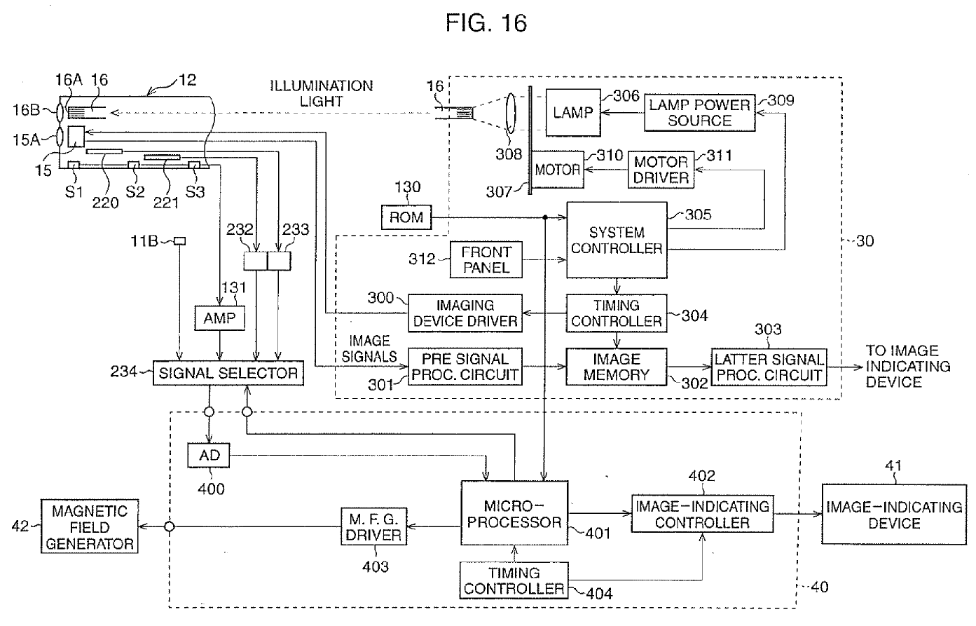

FIG. 16 is a block diagram that shows the electrical structure of the electronic endoscope system of the second embodiment.

Signals from the magnetic sensor coils S1-Sn are fed to a signal selector 234 that is provided inside the connector 13A (see FIG. 1) via the multi-channel amplifier 131. Further, variations in the electrical resistance of the strain gauges 220 and 221 are detected by strain gauge circuits 232 and 233 that may be provided inside the connector 13A. The signals from the strain gauge circuits 232 and 233 are then fed to the signal selector 234 as well as the signals from the coils S1-Sn. Further, signals from the angle lever sensor 111 are also fed to the signal selector 234, inside the connector 13A, via the light guide cable 13 (see FIG. 1).

The signals selector 234 is a circuit that is for selectively outputting the signals from the coils S1-Sn, the signals from the strain gauges 220 and 221, and the signals from the angle lever sensor 11B, in a predetermined sequence. The signals output from the signal selector 234 are then fed to the A/D converter 400 inside the insertion-portion-shape monitoring unit 40, so that the signals are converted from analog signals to digital signals and then input to the microprocessor 401. The selection of signals that are output from the signal selector 234, and the timing of switching the selection, are controlled by control signals from the microprocessor 401 of the insertion-portion-shape monitoring unit 40.

In the microprocessor 401, the positions of the coils S1-Sn are calculated from the signals from the coils S1-Sn, as in the first embodiment. Further, the degree of strain generated in the strain gauges 220 and 221 is calculated based on the signals from the strain gauges 220 and 221.

Image data for representing the entire shape of the insertion portion 12 are generated at an image-indicating controller 402, based on the positional data of the coils S1-Sn, which are calculated by the microprocessor 401, the data detected by the strain gauges 220 and 221, and the signal from the angle lever sensor 11B. The signals of the image data are then fed to the image-indicating device 41, and the shape of the insertion portion 12 is represented on the image-indicating device 41 in the same way as in the first embodiment.

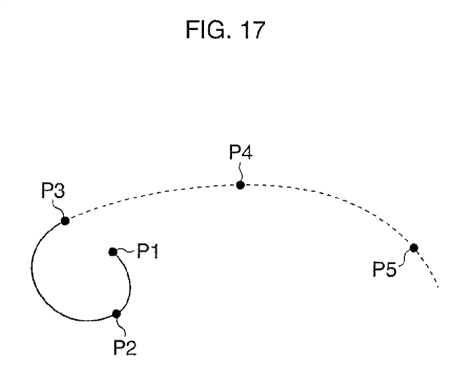

FIG. 17 schematically illustrates positions P1-P5 of the coils S1-S5 and an interpolation curve suitably applied to the positions PI-P5, when the angle lever 11A is operated and the bendable portion 200 is bent in a narrow arc, such that end face of the distal and portion 12C is turned around by approximately 270 degrees.

In FIG. 17, sections that correspond to the bendable portion 200 are indicated by a solid line, and sections that correspond to the flexible portion 12A are indicated by a phantom line. As described in the first embodiment, the flexible portion 12A can be accurately represented by connecting the points P3-Pn, which correspond to the flexible portion 12A, with a Bézier curve or a spline curve, while the bendable portion 200 cannot be appropriately represented in the same way.

In the second embodiment, positions of both ends of the bendable portion 200 and at least one position of a point within the bendable portion 200 are detected. Further, the degree of bending, which is defined in intervals between the above-detected points for each section is detected per section. Based on the above positional data and bending information, the shape of the bendable portion 200 is more precisely determined, and the precise shape of the bendable portion 200 is represented by the image-indicating device 41, as shown in FIG. 17.

Note that the bending properties of the bendable portion 200 are usually specific for each product. Therefore, in the second embodiment, correspondences between the output from the strain gauges 220 and 221 and information that represents the bending shape of the corresponding section, such as the curvature, are stored in the ROM 130 for each endoscope, for example, in a lookup table.

In the microprocessor 401, the degree of bending of each section, such as the curvature, is obtained by signals from the strain gauges 220 and 221, based on data stored in the ROM 130. Namely, the curvatures of the sections S1-S2 and S2-S3 of the bendable portion 200, the positions of the points P1, P2, and P3, and the bending direction of the bendable portion 200 are determined, so that the shape of the bendable portion 200 can be reproduced accurately.

As in the first embodiment, the correspondence between the electrical resistance R of the strain gauges 220 and 221 and the curvature ρ of the bendable portion 200 are measured beforehand, and the information thereof is stored in the ROM 130 before shipment.

As described above, according to the second embodiment, the same effect as in the first embodiment is 15 obtained. Further, in the second embodiment, since the plurality of bending sensors and at least one position within the bendable portion are detected, the shape of the bendable portion can be more precisely determined.

Note that in the second embodiment, the number of coils provided within the bendable portion may also be a plurality. Further, the number of bending sensors (strain gauges) may also be greater than two.

In the first and second embodiments, although the correspondence between the electrical resistances of the strain gauges and the curvatures is provided in a memory inside the endoscope connector, it may also be stored in the memory provided inside the processor apparatus or a computer system combined with the endoscope system. In such a case, the data may be stored in the memory based on the type (for every model number) of the endoscope. The model numbers of the endoscope may be listed on the screen, and the data may be obtained by selecting a corresponding model number from the list. Further, the model number may be stored in the memory of the endoscope, and the data, which correspond to the model number, may be automatically selected from the memory provided on a device other than the endoscope.

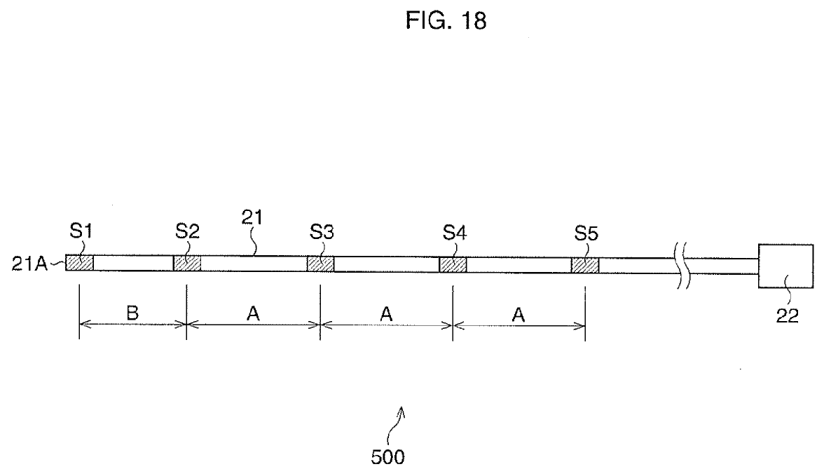

Next, with reference to FIGS. 18-20, a third embodiment of the endoscope-shape monitoring system of the present invention is explained below. The explanations will mainly be given for the structures that are dissimilar from the first and second embodiments. Further, the same reference numerals will be used for the same structures, as those in the first and second embodiments.

FIG. 18 schematically illustrates structures of a sensor unit used in the endoscope-shape monitoring system of the third embodiment.

In the third embodiment, the sensor unit is formed as a detachable type unit. The sensor unit 500 comprises a flexible tube 21 and a connector 22 that is attached on a proximal end of the flexible tube 21.

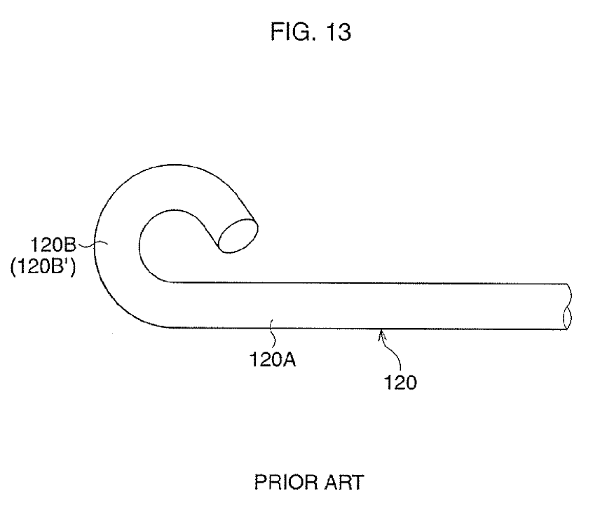

For example, the length of the flexible tube 21 is approximately equal to the sum of the length of an insertion portion 12, of an endoscope and the length of the light guide cable 13 (see FIG. 13. The distal end 21A of the flexible tube 21 is inserted into an instrument channel of the endoscope through the instrument channel opening 11C (see FIG. 1), so that the distal end 21A of the flexible tube 21 is arranged at the distal end of the instrument-channel, which is positioned in the distal end portion 12C of the endoscope.

Here, the instrument-channel is a conduit that is formed inside the insertion portion 12′, from the operating portion 11 to the distal end portion 12C. Namely, the instrument channel opening 11C is provided on the operating portion 11.

The first coil S1 is provided on the distal end 21A of the flexible tube 21. The second coil S2 is disposed inside the flexible tube 21 at a position separated from the coil 31 by a distance “B” along the axis of the flexible tube 21. Further, the coils S3, S4, S5, . . . , Sn are successively arranged at the predetermined intervals A, from the side of the coils S2 to the side of the connector 22. The coils S1-Sn are electrically connected to the connector 22.

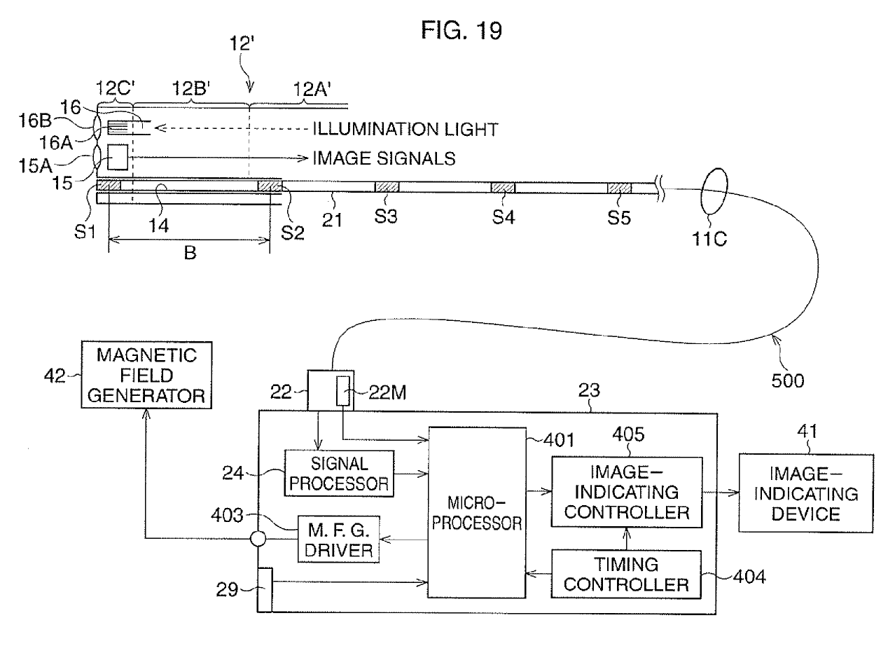

FIG. 19 is a block diagram that schematically illustrates the endoscope-shape monitoring system of the third embodiment. The endoscope-shape monitoring system of the third embodiment comprises the detachable sensor unit 500, a position detector 23 (corresponding to the insertion-portion-shape monitoring unit 40), the magnetic field generator 42, and the image-indicating device 41 In FIG. 19, the flexible tube 21 of the detachable sensor unit 500 is suitably installed in the instrument channel of the endoscope. Namely, the detachable sensor unit 500 is inserted into the instrument channel 14 of the insertion portion 12′ through the instrument channel opening 11C, and the distal end of the flexible tube 21 is positioned at the distal end portion 12C of the insertion portion 12′. Therefore, the coil S1 is disposed at the distal end portion 12C.

In the third embodiment, the distance B is slightly greater than the length of the bendable portion 12B′, so that when the installation of the sensor unit 500 into the instrument channel completes, the sensor S1 is disposed at the distal end portion 12C, the sensor S2 at the front end of the flexible portion 12A′, and the sensors S3-Sn in the flexible portion 12A′.

The connector 22 of the sensor unit 500 is detachably connected to the position detector 23. Signals from the coils S1-Sn of the sensor unit 500 are fed to a signal processor 24 inside the position detector 23. At the signal processor 24, the signals from the coils S1-Sn are subjected to amplification, detection, and A/D conversion, and are fed to the microprocessor 401 of the position detector 23, Further, a non-volatile memory 22M is provided in the connector 22. When the connector 22 is attached to the position detector 23, the memory 22M is electrically connected to the microprocessor 401. As is detailed below, data (bendable-portion shape data) that are used for representing the shape of the bendable portion 12B′, when the insertion-portion shape-indicating process is carried out, are stored in the memory 22M. The bendable-portion shape data are transmitted from the memory 22M to the microprocessor 401 when the endoscope-shape monitoring system is powered on, and the connector 22 is attached to the position detector 23.

As shown in FIG. 9, the positions of the point P1 in each of the above nine bending situations are represented by P1(0)-P1(8). Further, the direction of the distal end portion 12C′ when the bendable portion 12B, is being bent is represented by an angle “θ”, where the angle “θ” represents an angle against the direction of the distal end portion 12C′, when the bendable portion 12B′ is directed straight forward and is not bent. Thus, the bending situation is represented by the angle “θ”. Namely, when the bendable portion 12B′ is not bent and the point P1 is positioned at P1(0), the angle θ=0°. Further, when the bendable portion 12B′ is bent such that the distal end portion 12C′ faces in the opposite direction, and when the point P1 is positioned at P1(8), the angle θ=180°. Moreover, the angles “θ” for each of the positions P1(0)-P1(8) are represented by θ0-θ8.

In general, the distance “D” between the point P1 and the point P2 and the angle “θ” have a one-to-one correspondence (i.e., D=D(θ), θ=D⁻¹(D)). Further, when the distal end portion 12C′ is directed in a certain direction “θ”, the bendable portion 12B′ generally describes the same shape. Therefore, when the distance “D” is determined from the positions of the points P1 and P2, the shape of the bendable portion 12B′ can be determined.

In the third embodiment, a sensor unit 500 is provided that is adjusted for each endoscope. Information representing the correspondence between the distance IDC, (the relative distance between the points P1 and P2) and the shape of the bendable portion 12B′ is stored in the memory 22M inside the connector 22 of the sensor unit 500, as bendable-portion shape data. Note that the shapes of the bendable portion 12B′ that correspond to the distances “D” are measured beforehand and the distance “D” is calculated (determined) by the microprocessor 401 in accordance with the positions of the points P1 and P2. Examples of bendable-portion shape data are shown in Table 1.

| | | | | | | | | | |
|---|---|---|---|---|---|---|---|---|---|
| P1 (0) | X1, Y1, Z1 | X2, Y2, Z2 | X3, Y3, Z3 | X4, Y4, Z4 | X5, Y5, Z5 | X6, Y6, Z6 | X7, Y7, Z7 | X8, Y8, Z8 | X9, Y9, Z9 |
| P1 (1) | X1, Y1, Z1 | X2, Y2, Z2 | X3, Y3, Z3 | X4, Y4, Z4 | X5, Y5, Z5 | X6, Y6, Z6 | X7, Y7, Z7 | X8, Y8, Z8 | X9, Y9, Z9 |
| P1 (2) | X1, Y1, Z1 | X2, Y2, Z2 | X3, Y3, Z3 | X4, Y4, Z4 | X5, Y5, Z5 | X6, Y6, Z6 | X7, Y7, Z7 | X8, Y8, Z8 | X9, Y9, Z9 |
| P1 (3) | X1, Y1, Z1 | X2, Y2, Z2 | X3, Y3, Z3 | X4, Y4, Z4 | X5, Y5, Z5 | X6, Y6, Z6 | X7, Y7, Z7 | X8, Y8, Z8 | X9, Y9, Z9 |
| P1 (4) | X1, Y1, Z1 | X2, Y2, Z2 | X3, Y3, Z3 | X4, Y4, Z4 | X5, Y5, Z5 | X6, Y6, Z6 | X7, Y7, Z7 | X8, Y8, Z8 | X9, Y9, Z9 |
| P1 (5) | X1, Y1, Z1 | X2, Y2, Z2 | X3, Y3, Z3 | X4, Y4, Z4 | X5, Y5, Z5 | X6, Y6, Z6 | X7, Y7, Z7 | X8, Y8, Z8 | X9, Y9, Z9 |
| P1 (6) | X1, Y1, Z1 | X2, Y2, Z2 | X3, Y3, Z3 | X4, Y4, Z4 | X5, Y5, Z5 | X6, Y6, Z6 | X7, Y7, Z7 | X8, Y8, Z8 | X9, Y9, Z9 |
| P1 (7) | X1, Y1, Z1 | X2, Y2, Z2 | X3, Y3, Z3 | X4, Y4, Z4 | X5, Y5, Z5 | X6, Y6, Z6 | X7, Y7, Z7 | X8, Y8, Z8 | X9, Y9, Z9 |
| P1 (8) | X1, Y1, Z1 | X2, Y2, Z2 | X3, Y3, Z3 | X4, Y4, Z4 | X5, Y5, Z5 | X6, Y6, Z6 | X7, Y7, Z7 | X8, Y8, Z8 | X9, Y9, Z9 |

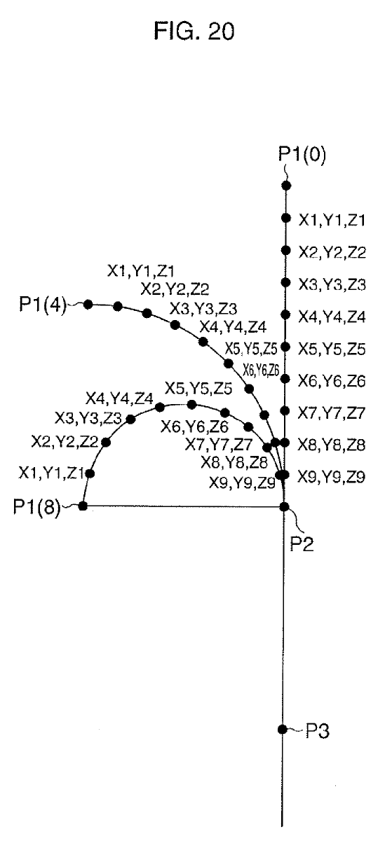

As shown in Table 1, the bendable-portion shape data, for example, include coordinates (x,y,z) of positions that are allocated along the central axis of the bendable portion 12B′ per a predetermined interval for each of the relative positions P1(0)-P1(8). As for the examples shown in Table 1, the positional coordinate data for the bendable portion 12B′ between the points P1 and P2 are given so that the interval between the points P1 and P2 is evenly divided into ten intervals. For each of the points P1(0)-P1(8), nine positional coordinate data (X1,Y1,Z1)-(X9,Y9,Z9) are stored. The correspondence between the positional coordinate data (X1,Y1,Z1)-(X9,Y9,Z9) and the bendable portion 12B′ is schematically illustrated in FIG. 20, for the situations where the point P1 is positioned at P1(0), P1(4), and P1(8).

As mentioned above, when the distance “D” is calculated, the position of the point P1 with respect to the point P2 is uniquely determined (the degree of freedom about the axis is not considered). Thereby, one of the positions P1(0)-P1(8) is selected in accordance with the determination, and the shape of the bendable portion 12B′ is reproduced based on positional coordinate data (X1,Y1,Z1)-(X9,Y9,Z9) corresponding to the selected position.

The bendable-portion shape data in the present embodiments may be positional information relating to any predetermined positions between the points P1 and P2, and the information may also include the curvature of the bendable portion 12B′ for each situation. Further, an interpolation function or parameters thereof may also be used for reproducing the shape of the bendable portion 12B′, so that the information of the interpolation function and the parameters may be stored in the memory for each of the distances “D”. Moreover, any combinations of the above methods may also be adopted.

Namely, in the insertion-portion shape-indicating process of the present embodiments, different interpolation methods are applied for each of the bendable portion 12B′ and the flexible portion 12A′, so that the entire shape of the insertion portion 12′ is represented by the combination thereof. Namely, regarding the flexible portion 12A′, each position of the coils is represented by a Bézier curve or a spline curve, in the same way as conventionally. On the other hand, regarding the bendable portion 12B′ and the distal end portion 12C′, the shape is represented by the interpolation based on the given insertion-portion shape data and the relative positional relationship between the coils S1 and S2, which are provided on both ends of the bendable portion 12B′, such as on the flexible portion 12A′ side and on the distal end portion 12C′ side.

Note that, when the Bézier curve or the spline curve is used to represent the flexible portion 12A′, a control point for the point P2 of the interpolation curve of the flexible portion 12A′ is determined from the geometrical parameters, such as for the tangential line and the curvature, selected for the bendable portion 12B′.

As described above, according to the third embodiment, in addition to the effects mentioned in the first and second embodiments, the shape of the bendable portion can be accurately obtained without using a bending sensor. Further, since the separate sensor unit, which is detachable from the instrument channel, is used, the system of the third embodiment can be applied for any conventional endoscope.

In the third embodiment, the position detector is used to obtain the data for representing the shape of the insertion portion, and the image-indicating device is directly connected to the position detector. However, the positional data of the coils may be transmitted to an external computer system, and the shape of the insertion portion may be represented on a screen of the computer system.

Further, in the third embodiment, the situation of the bendable portion is assumed to be uniquely determined by the distance between the coils S1 and S2, so that only the above distance is used to determine the condition or shape of the bendable portion, and the corresponding bendable-portion shape data are referenced. However, the directions of the coils may also be used to determine the situation of the bendable portion, if differences among the above distances are not sufficient to determine the situation.

In the third embodiment, although the bendable-portion shape data are stored in the memory inside the connector of the sensor unit, it may also be stored in a memory provided inside the processor apparatus or a computer system combined with the endoscope system. In such a case, the data may be stored in the memory based on the type (for every model number) of the sensor unit or the endoscope. The model numbers of the sensor unit or the endoscope may be listed on the screen, and the data may be obtained by selecting a corresponding model number from the list. Further, the model number may be stored in the memory of the sensor unit, and the bendable-portion shape data, which correspond to the model number, may be automatically selected from a memory provided on a device other than the sensor unit.

In the present embodiments, an alternating magnetic field is generated outside the endoscope, by the magnetic field generator disposed outside an inspection object, and the coils and the magnetic sensors are disposed inside the insertion portion. However, the coils for generating a magnetic field may be disposed inside the insertion portion, and magnetic sensors may be disposed outside the insertion portion.

Although the embodiment of the present invention has been described herein with reference to the accompanying drawings, obviously many modifications and changes may be made by those skilled in this art without departing from the scope of the invention.

The present disclosure relates to subject matter contained in Japanese Patent Applications Nos. 2005-324805, 2005-325226, and 2005-324935 (each filed on Nov. 9, 2005), which are expressly incorporated herein, by reference, in their entirety.

1. An endoscope shape monitoring system that is used to grasp a shape of a flexible insertion portion, the system comprising:
a position detecting system that detects positions of both ends of a bendable portion of said insertion portion;
a bending determinator that determines a bending situation of said bendable portion; and
a bendable-portion-shape reproducing processor that reproduces the shape of said bendable portion in accordance with said positions and said bending situation.

2. The system as claimed in claim 1, further comprising a bending direction detector that detects a bending direction of said bendable portion.

3. The system as claimed in claim 2, wherein said bending direction detector is provided on an angular lever of said endoscope, and comprises a sensor for detecting a direction of the angle lever operation.

4. The system as claimed in claim 1, wherein said position detecting system employs an alternating magnetic field.

5. The system as claimed in claim 4, wherein said position detecting system comprises a magnetic field generator that generates said alternating magnetic field, and a plurality of magnetic sensors for detecting said alternating magnetic field, and said plurality of magnetic sensors are disposed inside said insertion portion.

6. The system as claimed in claim 5, further comprising
a bending direction detector that detects a bending direction of said bendable portion;
a strain gauge that extends along said bendable portion;
a signal selector that selectively outputs signals from said plurality of magnetic sensors, said bending direction detector, and said strain gauge; and
an A/D converter that converts the signals that are output from said signal selector from analog to digital format.

7. The system as claimed in claim 5, wherein two of said plurality of magnetic sensors are disposed on said both ends of said bendable portion, and a first magnetic sensor is disposed on a distal end side of said insertion portion, and a second magnetic sensor is disposed on a flexible portion side of said insertion portion.

8. The system as claimed in claim 1, wherein said bending determinator comprises a strain gauge that extends along said bendable portion.

9. The system as claimed in claim 3, further comprising a memory that stores correspondence between output from said strain gauge and a curvature of said bendable portion, and said bendable-portion-shape reproducing processor reproduces the shape of said bendable portion in accordance with said curvature.

10. The system as claimed in claim 9, wherein said memory is provided in a connector of said endoscope.

11. The system as claimed in claim 1, further comprising a flexible-portion-shape reproducing processor that reproduces the shape of a flexible portion of said insertion portion in a way different from that carried out in said bendable-portion-shape reproducing processor.

12. The system as claimed in claim 1, wherein said position detecting system further detects a position of at least one point within said bendable portion, and said bending determinator determines bending situations at a plurality of positions in said bendable portion, so that said bendable-portion-shape reproducing processor reproduces the shape of said bendable portion in accordance with said positions and said bending situations.

13. The system as claimed in claim 1, further comprising:
a distance detector that detects the distance between said both ends of said bendable portion in accordance with said positions of said both ends; and
a memory that stores bendable-portion shape data corresponding to the distance, so that said bending situation of said bendable portion is determined from the distance, and said bendable-portion-shape reproducing processor reproduces the shape of said bendable portion in accordance with said bendable-portion shape data.

14. The system as claimed in claim 13, wherein said position detecting system comprises a magnetic field generator that generates said alternating magnetic field, and a plurality of magnetic sensors for detecting said alternating magnetic field, and said plurality of magnetic sensors are arranged in a detachable sensor unit that is formed as a flexible tube, said flexible tube being detachably inserted into a predetermined channel so that two of said magnetic sensors are disposed at said both ends of said bendable portion.

15. The system as claimed in claim 14, wherein said memory is provided on said detachable sensor unit.

16. The system as claimed in claim 13, wherein said bendable-portion shape data comprise positional information of at least one point of said bendable portion other than points on said both ends, and said positional information is given for each said bending situation.

17. An endoscope shape monitoring system that is used to grasp a shape of a flexible insertion portion, the system comprising:
a distance detector that detects a distance between both ends of a bendable portion of said insertion portion; and
a memory that stores bendable-portion shape data for reproducing the shape of said bendable portion in accordance with the distance.

18. The system as claimed in claim 17, wherein said distance detector comprises a position detector that detects positions of said both ends of said bendable portion, and a distance calculator that calculates said distance based on said positions of said both ends; and
said position detector comprises a magnetic field generator that generates said alternating magnetic field, a sensor unit that detects said alternating magnetic field, and a position calculator that calculates said positions of said both ends based on signals from said sensor unit.

19. The system as claimed in claim 18, wherein a first coil and a second coil are disposed, respectively, on said both ends.

20. The system as claimed in claim 19, wherein said sensor unit is formed as a flexible tube, said flexible tube being detachably inserted into a predetermined channel so that said first and second coils are disposed at said both ends of said bendable portion.

21. The system as claimed in claim 17, further comprising a bendable-portion-shape reproducing processor that reproduces the shape of said bendable portion in accordance with said bendable-portion shape data, and a flexible-portion-shape reproducing processor that reproduces the shape of a flexible portion of said insertion portion, further, said flexible-portion-shape reproducing processor represents the shape of said flexible portion by an interpolation curve that connects the positions of a plurality of sensors that are arranged along the axis of said flexible portion.
